(19) United States
(12) Patent Application Publication
(10) Pub. No.: US 2024/0336616 A1
(43) Pub. Date: Oct. 10, 2024

(54) MIDAZOLAM-D3 AND ITS PREPARATION METHOD

(71) Applicants: SHANGHAI RESEARCH INSTITUTE OF CRIMINAL SCIENCE AND TECHNOLOGY, Shanghai (CN); SHANGHAI YUANSI STANDARD SCIENCE AND TECHNOLOGY CO., LTD, Shanghai (CN)

(72) Inventors: Pingyong Liao, Shanghai (CN); Wenbin Liu, Shanghai (CN); Xuejun Zhao, Shanghai (CN); Wenbin Shao, Shanghai (CN); Shan He, Shanghai (CN); Jianwen Hu, Shanghai (CN); Yun Lan, Shanghai (CN); Junchang Wang, Shanghai (CN); Ruijia Chen, Shanghai (CN); Xilong Chen, Shanghai (CN)

(73) Assignees: SHANGHAI RESEARCH INSTITUTE OF CRIMINAL SCIENCE AND TECHNOLOGY, Shanghai (CN); SHANGHAI YUANSI STANDARD SCIENCE AND TECHNOLOGY CO., LTD, Shanghai (CN)

(21) Appl. No.: 18/589,554

(22) Filed: Feb. 28, 2024

(30) Foreign Application Priority Data
Apr. 4, 2023 (CN) 202310352682.1

(51) Int. Cl. C07D 487/04

(52) U.S. Cl. CPC C07D 487/04

(57) ABSTRACT

A compound midazolam-D3 and its preparation method as it's synthesized from 7-chloro-5-(2-fluorophenyl)-1,3-dihydro-2H-1,4-benzodiazepin-2-one (Compound II), by ring closure reaction with ethyl isocyanoacetate, hydrolysis with base, ring-opening with acid, ring closure under high temperature, and reacted with trideuteromethyl reagent; the synthetic route has advantage of short synthetic route appropriate, accessible and affordable. The prepared midazolam-D3 has characteristic with high purity, high deuterium content and very good stability.

### CROSS-REFERENCE TO RELATED APPLICATIONS

This application claims priority to Chinese Patent Application Ser. No. CN202310352682.1 filed on 4 Apr. 2023.

### TECHNICAL FIELD

The invention relates to a technique in the field of organic synthesis, in particular to midazolam-D3 and its preparation method.

### BACKGROUND OF THE INVENTION

For a long time, the deuterated internal standard used in our country depends on import, because the deuterated internal standard is too expensive, it was not widely used in our country. Based on survey and research on related literatures, there is no literature on the synthesis of deuterated midazolam-D3 at present, and it is also urgent to develop a method for the preparation of deuterated midazolam-D3 which is appropriate, accessible and affordable.

### SUMMARY OF THE INVENTION

The invention overcome the deficiencies and fill the research blank of midazolam-D3 and its preparation, which has a short synthetic route, appropriate, accessible and affordable. The prepared midazolam-D3 has the advantages of high purity, high deuterium content and good stability.

The present invention is achieved by the following method:

The present invention relates to midazolam-D3 (Compound I), having the chemical structure of

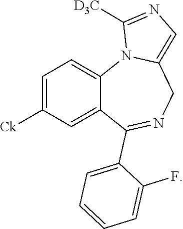

The present invention relates to the preparation of midazolam-D3 as described above, synthesized from 7-chloro-5-(2-fluorophenyl)-1,3-dihydro-2H-1,4-benzodiazepin-2-one (Compound II), by ring closure reaction with ethyl isocyanoacetate, hydrolysis with base, ring-opening with acid, ring closure under high temperature, and reacted with trideuteromethyl reagent, which specifically includes:

Step 1) 7-chloro-5-(2-fluorophenyl)-1,3-dihydro-2H-1,4-benzodiazepin-2-one (Compound II) was reacted with base and then chlorophosphonate was added to the reaction mixture, after that base and ethyl isocyanoacetate was added to give ethyl 8-chloro-6-(2-fluorophenyl)-4H-benzo[f]imidazo[1,5-a][1,4]diazepine-3-carboxylate (Compound III);

The said chlorophosphonate is dimethyl phosphorochloridate, diethyl phosphorochloridate, dipropyl phosphorochloridate, dimorpholinophosphinyl chloride, or its mixture.

The said base is NaH, n-BuLi, t-BuOK, t-BuONa, t-BuMgCl, LiHMDS, NaHMDS, KHMDS, EtMgBr, or its mixture.

Step 2) Ethyl 8-chloro-6-(2-fluorophenyl)-4H-benzo[f]imidazo[1,5-a][1,4]diazepine-3-carboxylate (Compound III) was hydrolysis with base in the solvent to give 8-chloro-6-(2-fluorophenyl)-4H-benzo[f]imidazo[1,5-a][1,4]diazepine-3-carboxylic acid (Compound IV);

The reaction solvent is MeOH, EtOH, n-PrOH, THF, H₂O, i-PrOH, 1,4-dioxane or its mixture.

The said base is LiOH, NaOH, KOH, EtONa or its mixture.

Step 3) 8-chloro-6-(2-fluorophenyl)-4H-benzo[f]imidazo[1,5-a][1,4]diazepine-3-carboxylic acid (Compound IV) was dissolved in solvent and ring closure under acid condition to give 5-(aminomethyl)-1-{4-chloro-2-[(2-fluorophenyl) carbonyl]phenyl}-1H-imidazole-4-carboxylic acid (compound V).

The reaction solvent is MeOH, EtOH, n-PrOH, THF, H₂O, i-PrOH, 1,4-dioxane, DMSO, DMF or its mixture.

The said acid is H₂SO₄, HBr, HCl, CF₃COOH, CH₃SO₃H, CH₃COOH, CF₃SO₃H or of its mixture.

Step 4) 5-(aminomethyl)-1-{4-chloro-2-[(2-fluorophenyl) carbonyl]phenyl}-1H-imidazole-4-carboxylic acid (compound V) dissolved in solvent and through decarboxylation under high temperature to obtain desmethylmidazolam (compound VI).

The reaction solvent is DMAc, toluene, 1,4-dioxane, decahydronaphthalene, HMPA, NMP, 1,3-dimethyl-2-imidazolidinone, DMSO or its mixture.

The reaction temperature is 100-200° C.

Step 5) To a solution of desmethylmidazolam (compound VI) in solvent, base and CD₃I was added, after purification to obtain midazolam-D3 (compound I).

The said base is NaH, MeMgBr, LiHMDS, NaHMDS, KHMDS, n-BuLi, LDA, t-BuOK, t-BuONa, t-BuMgCl or its mixture.

The reaction solvent is ether, MTBE, 1,4-dioxane, THF, 2-MeTHF, acetonitrile or its mixture.

### BRIEF DESCRIPTION OF DRAWINGS

FIG. 1 shows the 1H NMR spectrum of midazolam-D3 prepared in Example 1;

FIG. 2 shows the 13C NMR spectrum of midazolam-D3 prepared in Example 1;

FIG. 3 shows the HRMS spectrum of midazolam-D3 prepared in Example 1.

### DETAILED DESCRIPTION OF THE INVENTION

### Embodiment 1

This embodiment relates to a preparation method of midazolam-D3, specifically comprising:

Step 1) To a solution of 7-chloro-5-(2-fluorophenyl)-1,3-dihydro-2H-1,4-benzodiazo-2-one (compound II, 2.88 g, 10 mmol) in anhydrous THF (40 mL) was added LiHMDS (1.2 eq.) at ice bath. The temperature adjusted to −20 degrees after 20 min, and dimethyl chlorophosphate (1.5 eq.) was added slowly. After that, the reaction mixture was maintained at 0 degrees for 1 h; then added ethyl isocyanoacetate (1.5 eq.) and LiHMDS (1.5 eq.) at −20 degrees. Upon addition completed, it was reacted at room temperature for 4 h. After quenching with saturated sodium bicarbonate, the mixture was extracted thrice with dichloromethane, combined the organic phases, concentrated and purified on a flash column to obtain a yellow solid of 8-chloro-6-(2-fluorophenyl)-4H-benzo[f]imidazolo [1,5-a][1,4]diazo-3-carboxylic acid ethyl ester (compound III) with about 75% yield.

Step 2) 8-chloro-6-(2-fluorophenyl)-4H-benzo[f]imidazolo [1,5-a][1,4]diazo-3-carboxylic acid ethyl ester (compound III, 3.84 g, 10 mmol) from the previous step was dissolved in EtOH (40 mL) and 2 N LiOH (5 eq.) was added to the solution. The reaction mixture was stirred at room temperature for 4 h, and then adjusted to pH 3-4 with acetic acid. After stirring at ice bath for 0.5 h, the mixture was filtered and the solid was dried to afford 8-chloro-6-(2-fluorophenyl)-4H-benzo[f]imidazo[1,5-a][1,4]diazepine-3-carboxylic acid (Compound IV) as yellow solid with 92% yield.

Step 3) 8-chloro-6-(2-fluorophenyl)-4H-benzo[f]imidazo[1,5-a][1,4]diazepine-3-carboxylic acid (Compound IV, 3.56 g, 10 mmol) was dissolved in 2 N H₂SO₄ aqueous solution and stirred at room temperature for 4 h. The mixture was concentrated and the solid was precipitated out by adding EtOH. The resultant salt was filtered after 1-2 h stirring and dried to afford 5-(aminomethyl)-1-{4-chloro-2-[(2-fluorophenyl) carbonyl]phenyl}-1H-imidazole-4-carboxylic acid (compound V) as white product with 90% yield.

Step 4) 5-(aminomethyl)-1-{4-chloro-2-[(2-fluorophenyl) carbonyl]phenyl}-1H-imidazole-4-carboxylic acid (compound V, 3.74 g, 10 mmol) was dissolved in DMF (20 mL) and refluxed at 140 degrees for 12 h. The resulting solution was concentrated and adjusted to pH 11 by addition of saturated NaHCO₃. The solution was extracted with ethyl acetate several times. The combined organic phase was washed with water and concentrated to give crude product which was purified to afford desmethylmidazolam (compound VI) as yellow solid with 61% yield.

Step 5) To the solution of Desmethylmidazolam (compound VI, 3.12 g, 10 mmol) in anhydrous THF (100 mL), t-BuMgCl (1.2 eq.) was added dropwise at ice bath. After stirring at ice bath for 20 min, the CD₃I (1.5 eq.) was added and reacted at same temperature for 2 h. The reaction mixture was quenched and extracted with DCM thrice. The combined organic phase was concentrated and purified with flash column to obtain desired product midazolam-D3 (compound I) with 85% yield.

The synthetic route of this embodiment is as follows:

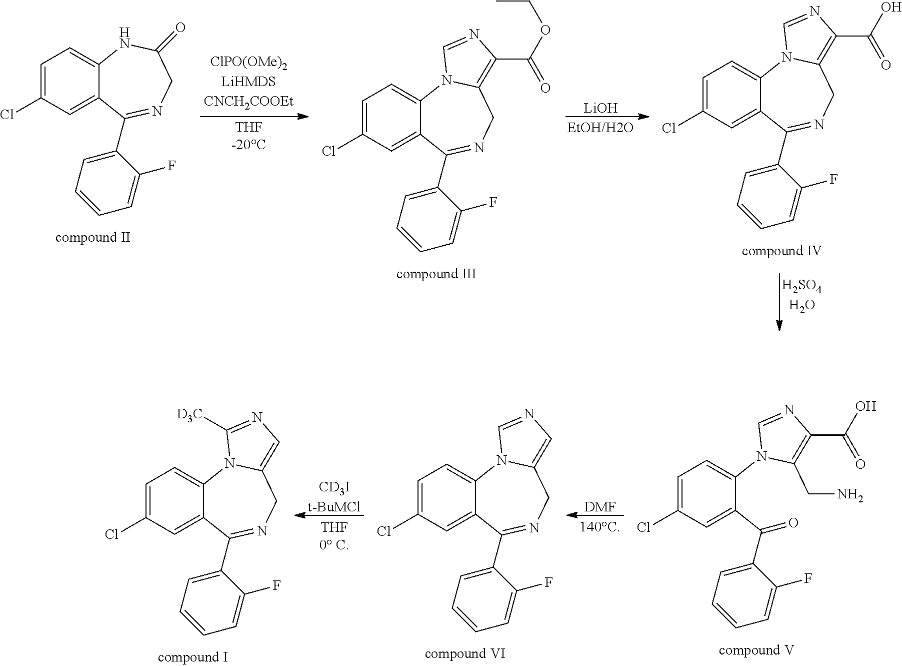

The midazolam-D3 (compound I) prepared in Embodiment 1 was characterized using 1H NMR, 13C NMR, HRMS, HPLC and deuterium content respectively. These data are as follows:

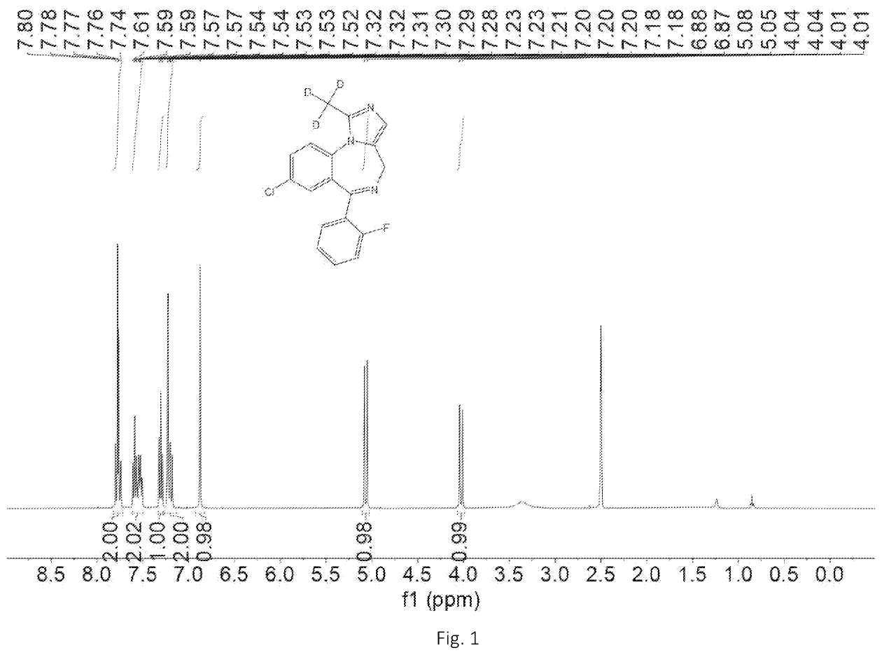

As shown in FIG. 1: ¹H NMR (400 MHZ, DMSO-d6) δ 7.80-7.74 (m, 2H), 7.61-7.51 (m, 2H), 7.30 (td, J=7.6, 1.1 Hz, 1H), 7.236-7.18 (m, 2H), 6.87 (d, J=0.9 Hz, 1H), 5.06 (d, J=12.9 Hz, 1H), 4.02 (d, J=12.9 Hz, 1H).

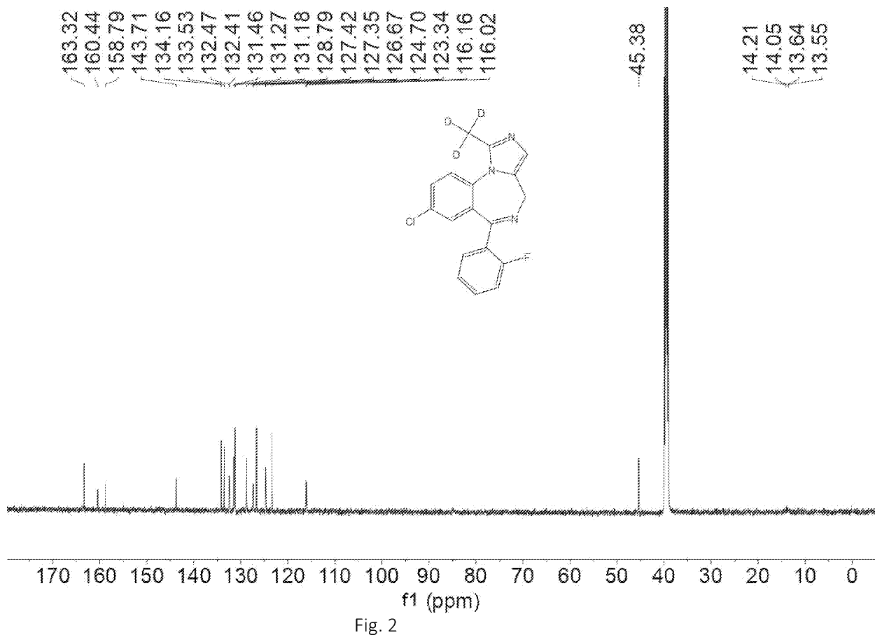

As shown in FIG. 2: ¹³C NMR (151 MHZ, DMSO-d6) δ 163.32, 160.44, 158.79, 143.71, 134.16, 133.53, 132.47, 132.41, 131.46, 131.27, 131.18, 128.79, 127.42, 127.35, 126.67, 124.70, 123.34, 116.16, 116.02, 45.38.

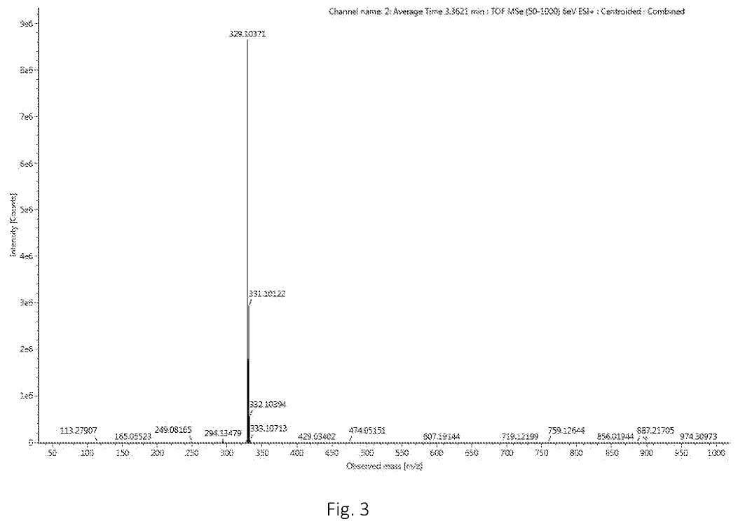

As shown in FIG. 3: HRMS (ESI) calcd. for C₁₈H₁₀D₃ClFN₃ [M+H]⁺329.10431, found: 329.10371.

As shown in table 1: The HPLC purity is 98.979%.

PDA CH1 210 nm

| | | | | | | | | |
|---|---|---|---|---|---|---|---|---|
| Peak | Retention time | Compound name | Area | Height | Area % | Resolution | Tailing factor | USP |

| | | | | | | | |
|---|---|---|---|---|---|---|---|
| 1 | 2.765 | 577 | 115 | 0.010 | — | 2.029 | 3825 |
| 2 | 7.030 | 10210 | 1138 | 0.175 | 19.677 | 1.811 | 12193 |
| 3 | 7.654 | 11557 | 1318 | 0.198 | 2.435 | 1.563 | 14166 |
| 4 | 7.990 | 2999 | 310 | 0.051 | 1.250 | 1.987 | 12964 |
| 5 | 8.969 | 1142 | 143 | 0.020 | 3.877 | 1.838 | 25476 |
| 6 | 9.535 | 5787393 | 613435 | 98.979 | 2.264 | 1.519 | 19273 |
| 7 | 10.076 | 15624 | 1363 | 0.267 | 1.718 | — | 12931 |
| 8 | 13.316 | 3259 | 459 | 0.056 | 10.844 | — | 47970 |
| 9 | 13.498 | 11897 | 1086 | 0.203 | 0.621 | — | 24791 |
| 10 | 15.385 | 2459 | 352 | 0.042 | 7.028 | 1.152 | 100352 |
| Total | | 5847115 | 619720 | 100.000 | | | |

As shown in table 2: The deuterium content is 99.41%.

| Name | Midazolam-D3 | Structure |
|---|---|---|
| Molecular formula Exact mass Deuterium content 1 Deuterium content 2 Deuterium content 3 Average of deuterium content RSD | C18H10D3ClFN3 328.09703 99.41% 99.41% 99.40% 99.41% 0.01% | 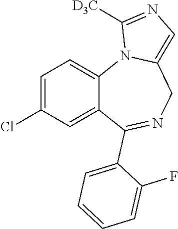 [SMILES: [2H]C([2H])([2H])c1ncc2n1-c1ccc(Cl)cc1C(c1ccccc1F)=NC2] |

### Embodiment 2

This embodiment relates to a preparation method of midazolam-D3, specifically comprising:

Step 1) To a solution of 7-chloro-5-(2-fluorophenyl)-1,3-dihydro-2H-1,4-benzodiazo-2-one (compound II, 2.88 g, 10 mmol) in anhydrous THF (40 mL) was added t-BuONa (1.2 eq.) at ice bath. The temperature adjusted to −20 degrees after 20 min, and dimethyl chlorophosphate (1.5 eq.) was added slowly. After that, the reaction mixture was maintained at 0 degree for 1 h; then added ethyl isocyanoacetate (1.5 eq.) and t-BuONa (1.5 eq.) at −20 degrees. Upon addition completed, it was reacted at room temperature for 4 h. After quenching with saturated sodium bicarbonate, the mixture was extracted thrice with dichloromethane, combined the organic phases, concentrated and purified on a flash column to obtain a yellow solid of 8-chloro-6-(2-fluorophenyl)-4H-benzo[f]imidazolo [1,5-a][1,4]diazo-3-carboxylic acid ethyl ester (compound III) with about 82% yield.

Step 2) 8-chloro-6-(2-fluorophenyl)-4H-benzo[f]imidazolo [1,5-a][1,4]diazo-3-carboxylic acid ethyl ester (compound III, 3.84 g, 10 mmol) from the previous step was dissolved in EtOH (40 mL) and 2 N KOH (10 eq.) was added to the solution. The reaction mixture was stirred at room temperature for 4 h, and then adjusted to pH 3-4 with acetic acid. After stirring at ice bath for 0.5 h, the mixture was filtered and the solid was dried to afford 8-chloro-6-(2-fluorophenyl)-4H-benzo[f]imidazo[1,5-a][1,4]diazepine-3-carboxylic acid (Compound IV) as yellow solid with 96% yield.

Step 3) 8-chloro-6-(2-fluorophenyl)-4H-benzo[f]imidazo[1,5-a][1,4]diazepine-3-carboxylic acid (Compound IV, 3.56 g, 10 mmol) was dissolved in mixture of THF (60 mL)/4 N HBr (10 eq.) aqueous solution and stirred at room temperature for 4 h. The mixture was concentrated and the solid was precipitated out by adding EtOH. The resultant salt was filtered after 1-2 h stirring and dried to afford 5-(aminomethyl)-1-{4-chloro-2-[(2-fluorophenyl) carbonyl]phenyl}-1H-imidazole-4-carboxylic acid (compound V) as white product with 88% yield.

Step 4) 5-(aminomethyl)-1-{4-chloro-2-[(2-fluorophenyl) carbonyl]phenyl}-1H-imidazole-4-carboxylic acid (compound V, 3.74 g, 10 mmol) was dissolved in DMI (20 mL) and reacted at 200 degrees for 3-4 h. The resulting solution was concentrated and adjusted to pH 11 by addition of saturated NaHCO₃. The solution was extracted with ethyl acetate several times. The combined organic phase was washed with water and concentrated to give crude product which was purified to afford desmethylmidazolam (compound VI) as yellow solid with 43% yield.

Step 5) To the solution of desmethylmidazolam (compound VI, 3.12 g, 10 mmol) in anhydrous 2-MeTHF (80 mL), LiHMDS (1.5 eq.) was added dropwise at −78 degrees. After stirring at −78 degrees for 1 h, the CD₃I (2 eq.) was added and reacted at same temperature for 2 h. The reaction mixture was quenched and extracted with DCM thrice. The combined organic phase was concentrated and purified with flash column to obtain desired product midazolam-D3 (compound I) with 75% yield.

The synthetic route of this embodiment is as follows:

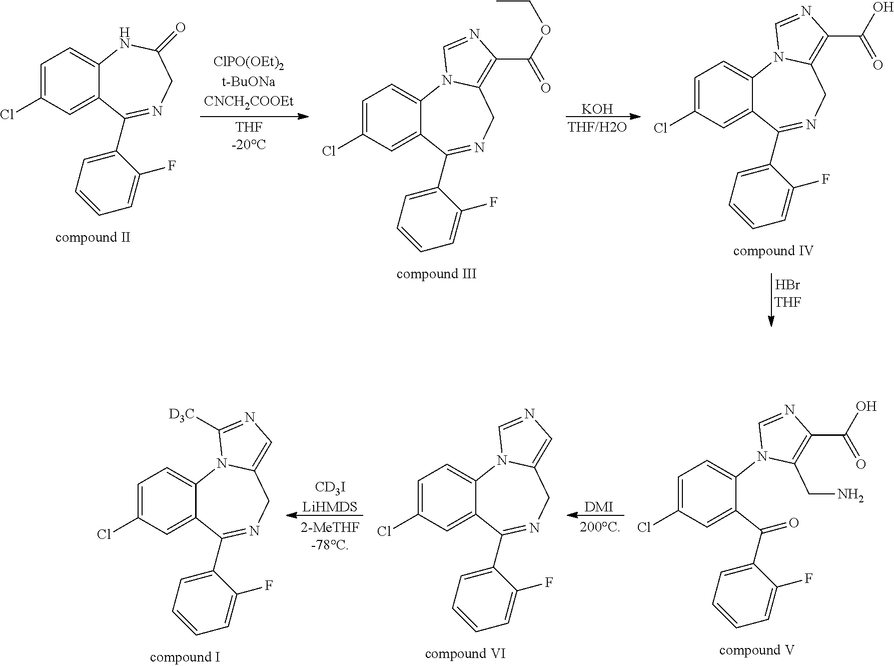

### Embodiment 3

This embodiment relates to a preparation method of midazolam-D3, specifically comprising:

Step 1) To a solution of 7-chloro-5-(2-fluorophenyl)-1,3-dihydro-2H-1,4-benzodiazo-2-one (compound II, 2.88 g, 10 mmol) in anhydrous THF (40 mL) was added t-BuMgCl (1.2 eq.) at ice bath. The temperature adjusted to −20 degrees after 20 min, and dimethyl chlorophosphate (1.5 eq.) was added slowly. After that, the reaction mixture was maintained at 0 degree for 1 h; then added ethyl isocyanoacetate (1.5 eq.) and t-BuMgCl (1.5 eq.) at −20 degrees. Upon addition completed, it was reacted at room temperature for 4 h. After quenching with saturated sodium bicarbonate, the mixture was extracted thrice with dichloromethane, combined the organic phases, concentrated and purified on a flash column to obtain a yellow solid of 8-chloro-6-(2-fluorophenyl)-4H-benzo[f]imidazolo [1,5-a][1,4]diazo-3-carboxylic acid ethyl ester (compound III) with about 88% yield.

Step 2) 8-chloro-6-(2-fluorophenyl)-4H-benzo[f]imidazolo [1,5-a][1,4]diazo-3-carboxylic acid ethyl ester (compound III, 3.84 g, 10 mmol) from the previous step was dissolved in 2 N KOH (20 mL). The reaction mixture was stirred at room temperature for 4 h, and then adjusted to pH 3-4 with acetic acid. After stirring at ice bath for 0.5 h, the mixture was filtered and the solid was dried to afford 8-chloro-6-(2-fluorophenyl)-4H-benzo[f]imidazo[1,5-a][1,4]diazepine-3-carboxylic acid (Compound IV) as yellow solid with 79% yield.

Step 3) 8-chloro-6-(2-fluorophenyl)-4H-benzo[f]imidazo[1,5-a][1,4]diazepine-3-carboxylic acid (Compound IV, 3.56 g, 10 mmol) was dissolved in mixture of EtOH (20 mL)/6 N HCl (10 eq.) aqueous solution and stirred at room temperature for 6-8 h. The mixture was concentrated and the solid was precipitated out by adding EtOH. The resultant salt was filtered after 1-2 h stirring and dried to afford 5-(aminomethyl)-1-{4-chloro-2-[(2-fluorophenyl) carbonyl]phenyl}-1H-imidazole-4-carboxylic acid (compound V) as white product with 85% yield.

Step 4) 5-(aminomethyl)-1-{4-chloro-2-[(2-fluorophenyl) carbonyl]phenyl}-1H-imidazole-4-carboxylic acid (compound V, 3.74 g, 10 mmol) was dissolved in DMSO (20 mL) and reacted at 190 degrees for 6-8 h. The resulting solution was concentrated and adjusted to pH 11 by addition of saturated NaHCO₃. The solution was extracted with ethyl acetate several times. The combined organic phase was washed with water and concentrated to give crude product which was purified to afford desmethylmidazolam (compound VI) as yellow solid with 79% yield.

Step 5) To the solution of desmethylmidazolam (compound VI, 3.12 g, 10 mmol) in anhydrous MTBE (100 mL), LDA (0.9 eq.) was added dropwise at −78 degrees. After stirring at −78 degrees for 20 min, the CD₃I (1.1 eq.) was added and reacted at same temperature for 4 h. The reaction mixture was quenched and extracted with DCM thrice. The combined organic phase was concentrated and purified with flash column to obtain desired product midazolam-D3 (compound I) with 81% yield.

The synthetic route of this embodiment is as follows:

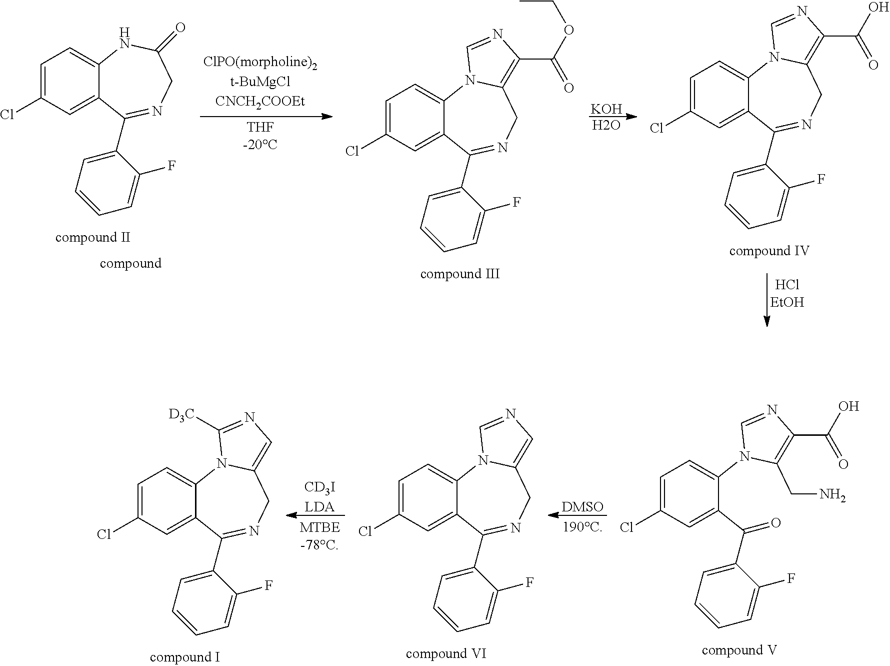

In summary, the prepared midazolam-D3 has characteristic with high purity, high deuterium content and good stability.

What is claimed is:

1. A compound midazolam-D3 having a structure shown as the following formula:

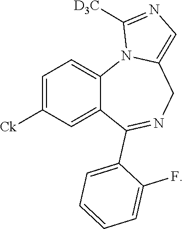

2. A method for the preparation of midazolam-D3 as claim 1, comprising the steps: 7-chloro-5-(2-fluorophenyl)-1,3-dihydro-2H-1,4-benzodiazepin-2-one (Compound II) as start material, by ring closure reaction with ethyl isocyanoacetate, hydrolysis with base, ring-opening with acid, ring closure under high temperature, and reacted with trideuteromethyl reagent.

3. A preparation method according to claim 2, comprising the steps:
step 1) 7-chloro-5-(2-fluorophenyl)-1,3-dihydro-2H-1,4-benzodiazepin-2-one (Compound II) was reacted with base and then chlorophosphonate was added to the reaction mixture, after that base and ethyl isocyanoacetate was added to give ethyl 8-chloro-6-(2-fluorophenyl)-4H-benzo[f]imidazo[1,5-a][1,4]diazepine-3-carboxylate (Compound III);
step 2) ethyl 8-chloro-6-(2-fluorophenyl)-4H-benzo[f]imidazo[1,5-a][1,4]diazepine-3-carboxylate (Compound III) was hydrolysis with base to give 8-chloro-6-(2-fluorophenyl)-4H-benzo[f]imidazo[1,5-a][1,4]diazepine-3-carboxylic acid (Compound IV);
Step 3) 8-chloro-6-(2-fluorophenyl)-4H-benzo[f]imidazo[1,5-a][1,4]diazepine-3-carboxylic acid (Compound IV) was dissolved in solvent and ring closure under acid condition to give 5-(aminomethyl)-1-{4-chloro-2-[(2-fluorophenyl) carbonyl]phenyl}-1H-imidazole-4-carboxylic acid (compound V);
step 4) 5-(aminomethyl)-1-{4-chloro-2-[(2-fluorophenyl) carbonyl]phenyl}-1H-imidazole-4-carboxylic acid (compound V) dissolved in solvent and through decarboxylation under high temperature to obtain desmethylmidazolam (compound VI);
step 5) to a solution of desmethylmidazolam (compound VI) in solvent, base and CD3I was added, after purification to obtain midazolam-D3 (compound I);
and the synthetic route of the described method is as:

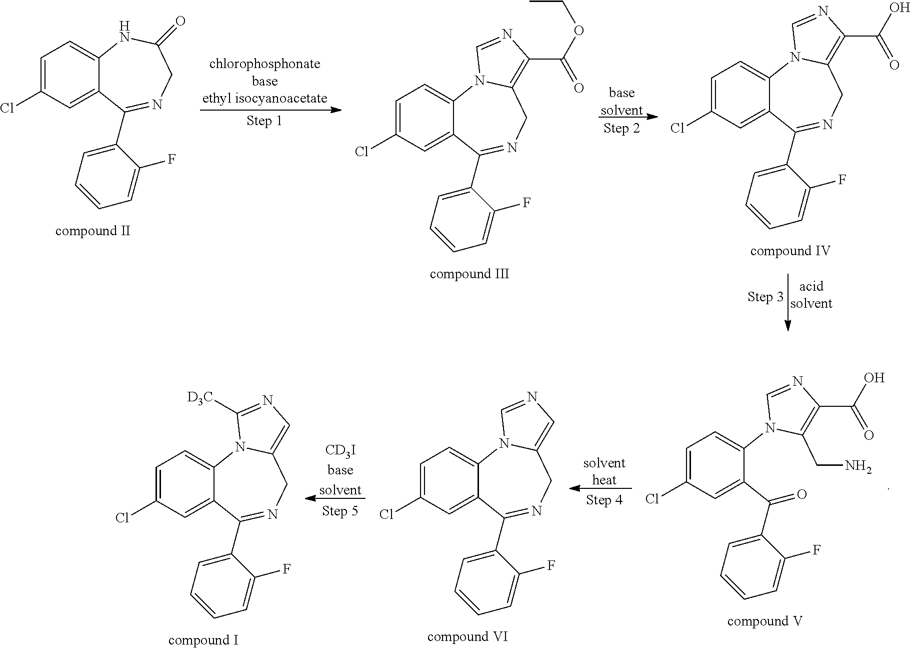

4. The preparation method according to claim 3, characterized in that: the chlorophosphonate in step 1 is one or more selected from the group consisting of dimethyl phosphorochloridate, diethyl phosphorochloridate and dipropyl phosphorochloridate, dimorpholinophosphinyl chloride, and
the base is one or more selected from the group consisting of NaH, n-BuLi, t-BuOK, t-BuONa, t-BuMgCl, LiHMDS, NaHMDS, KHMDS and EtMgBr.

5. The preparation method according to claim 3, characterized in that: the reaction solvent in step 2 is one or more selected from the group consisting of MeOH, EtOH, n-PrOH, THF, H₂O, i-PrO and, 1,4-dioxane, and
the base is one or more selected from the group consisting of LiOH, NaOH, KOH and EtONa.

6. The preparation method according to claim 3, characterized in that: the reaction solvent in step 3 is one or more selected from the group consisting of MeOH, EtOH, n-PrOH, THF, H₂O, i-PrOH, 1,4-dioxane, DMSO and DMF, and
the acid is one or more selected from the group consisting of H₂SO₄, HBr, HCl, CF₃COOH, CH₃SO₃H, CH₃COOH and CF₃SO₃H.

7. The preparation method according to claim 3, characterized in that: the reaction solvent in step 4 is one or more selected from the group consisting of DMAc, toluene, 1,4-dioxane, decahydronaphthalene, HMPA, NMP, 1,3-dimethyl-2-imidazolidinone and DMSO, and
the reaction temperature is 100-200° C.

8. The preparation method according to claim 3, characterized in that: the base in step 5 is one or more selected from the group consisting of NaH, MeMgBr, LiHMDS, NaHMDS, KHMDS, n-BuLi, LDA, t-BuOK, t-BuONa and t-BuMgCl, and
the reaction solvent is one or more selected from the group consisting of ether, MTBE, 1,4-dioxane, THF, 2-MeTHF and acetonitrile.
